(19) United States
(12) Patent Application Publication
(10) Pub. No.: US 2008/0102505 A1
(43) Pub. Date: May 1, 2008

(54) Method of treating viral infections with ultraviolet light

(76) Inventor: Thomas R. Petrie JR., Newfoundland, NJ (US)

Correspondence Address: MINTZ LEVIN COHN FERRIS GLOVSKY & POPEO, ONE FINANCIAL CENTER, BOSTON, MA, 02111, US

(21) Appl. No.: 11/901,806

(22) Filed: Sep. 18, 2007

Related U.S. Application Data
(60) Provisional application No. 60/845,384, filed on Sep. 18, 2006.
(60) Provisional application No. 60/845,348, filed on Sep. 18, 2006.
(60) Provisional application No. 60/880,663, filed on Jan. 16, 2007.

(51) Int. Cl. C12N 13/00

(52) U.S. Cl. 435/173.3

(57) ABSTRACT

A method of treating a viral infection by stimulating cell mediated immunity in a subject. Specifically, the instant method is performed by applying ultraviolet light to stimulating the subject's immune system to activate potent cell mediate immune responses, including inducing gene expression of various cytokines, against the specific viruses such as HIV and influenza.

### CROSS-REFERENCE TO RELATED APPLICATIONS

This application claims priority from U.S. Provisional Application Ser. No. 60/845,384, filed on Sep. 18, 2006, U.S. Provisional Application Ser. No. 60/880,663, filed on Jan. 16, 2007 and 60/845,348, filed on Sep. 18, 2006, the disclosures of which are incorporated herein by reference in their entireties.

This application also relates to U.S. patent application Ser. No. 11/441,547 to Petrie, filed May 26, 2006, entitled “Blood Irradiation System Device”, which claims priority to U.S. Provisional Patent Application No. 60/685,471 to Petrie, filed May 27, 2005, entitled “Blood Irradiation Device”, and which is a continuation-in-part application of U.S. patent application Ser. No. 11/285,959 to Petrie, filed Nov. 22, 2005, which claims priority to U.S. provisional application Nos. 60/630,503, filed Nov. 22, 2004 and 60/638,286, filed Dec. 21, 2004. The present application is also related to U.S. Pat. No. 6,312,593 to Petrie. The present application is also related to U.S. Parent Application No. 60/845,384 to Petrie, filed Sep. 18, 2006 and 60/845,348 to Petrie, filed Sep. 18, 2006. Each of the foregoing disclosures is herein incorporated by reference in their entirety.

Each of the applications and patents cited in this text, as well as each document or reference cited in each of the applications and patents (including during the prosecution of each issued patent; “application cited documents”), and each of the U.S. and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### FIELD OF THE INVENTION

The present invention relates in part to methods and systems for treating viral infections. Specifically, the present invention relates to an application of ultraviolet light (200 nm-400 nm) to treating infections caused by viral pathogenic agents, including RNA, DNA, episomal and integrative viruses.

### BACKGROUND OF THE INVENTION

Viral infections are associated with a significant economic burden on both society and the individual, resulting in considerable healthcare costs and loss of productivity, as well as intangible costs such as suffering, grief and social disruption. Despite years of research efforts, there remain no successful cure for many viruses, especially RNA viruses such as HIV and influenza.

Infection with the human immunodeficiency virus (HIV) results in progressive deterioration of the immune system in most infected subjects. During disease progression, key cells associated with the immune system become infected with HIV, including, e.g., T-lymphocytes (T-cells), and macrophages/monocytes. Prolonged HIV infection frequently culminates in the development of Acquired Immunodeficiency Syndrome (AIDS). In the late stages of this disease, the immune system is severely compromised due to loss or dysfunction of T cells (Shearer et al. (1991) AIDS 5:245 253).

HIV-1-specific cytotoxic T lymphocytes(CTL) appear to be critical in the immunologic control of HIV-1 soon after the acquisition of infection. CTL precursors specific for cells expressing several HIV-1 gene products, including Gag, Pol, and Env antigens, are detectable within three weeks of the primary infection syndrome (Koup et al. (1994) J. Virol. 68:4650 4655). Since CTL activity is antigen driven, the waning in responding T-cell subsets that generally occurs with the passage of time is not unexpected.

The clinical significance of this cellular immune response to HIV has been demonstrated in a number of studies, and impairment of this response appears to be associated with more rapid disease progression. Lymphokines elaborated by HIV-specific T-cells are critical in supporting the genesis of these mature cytotoxic T lymphocytes directed against HIV-1 (Rosenberg et al. (1997) Science 278:1447 1450), lending credence to the notion that virus-specific T-helper cells are necessary for maintenance of effective immunity to HIV.

Anti-retroviral drugs, such as reverse transcriptase inhibitors, viral protease inhibitors, and viral entry inhibitors, have been used to treat HIV infection (Caliendo et al. (1994) Clin. Infect. Dis. 18:516-524). More recently, treatment with combinations of these agents, known as highly active antiretroviral therapy (HAART), has been used to effectively suppress replication of HIV (Gulick et al. (1997) N. Engl. J. Med. 337:734-9); Hammer et al. (1997) N. Engl. J. Med. 337:725-733). However, HAART is primarily efficacious with regard to the prevention of the spread of infection into uninfected cells and this therapy cannot efficiently reduce the residual, latent proviral DNA integrated into the host cellular genome (Wong et al. (1997) Science 278:1291-1295; Finzi et al. (1997) Science 278:1295-1300 (see comments), Finzi et al. (1999) Nat. Med. 5:512-517; Zhang et al. (1999) N. Engl. J. Med. 340:1605-1613). Moreover, HAART is mostly focused on suppressing replication of the virus and not on the promoting immunological control of the HIV by enhancing host's cellular immune responses.

Thus, anecdotal reports of individuals who have discontinued HAART have revealed a rapid relapse of viremia, most often within a few weeks of ceasing anti-viral therapy (Ruiz et al. (2000) AIDS 14:397 403). Consequently, HAART must be administered indefinitely to prevent reactivation of latent virus. Continuous treatment with HAART is problematic, as HAART regimens are expensive, are difficult to comply with, and have many side effects. In addition, prolonged treatment with antiretroviral agents often leads to the emergence of drug resistant viral strains (Larder et al. (1989) Science 246:1155 1158; Kellam et al. (1992) Proc. Natl. Acad. Sci. USA 89:1934 1938; St. Clair et al. (1991) Science 253:1557 1559) and a significant portion of patients treated with combination therapy may eventually harbor strains of HIV having multi-drug resistance (Schinazi et al. (1994) Int. Antiviral News 2:72 5).

Influenza presents another example of a viral infection in great need of immunological control. The increased demand for influenza vaccines, coupled with the loss of vaccine manufacturers in the United States, has resulted in recurrent instances of shortages and delays in vaccine availability. Given the current manufacturing capabilities within the United States, experts estimate that if a pandemic does occur, it will take several months to a year to manufacture and distribute enough vaccine to those in need. Moreover, antiviral drugs such as Oseltamivir, which can treat most influenza infections, have shown only limited ability to control avian influenza virus replication, and an even lesser ability to control clinical illness and prevent death. Given the potential severe consequences of a global influenza outbreak, there is a need for the ability to modulate influenza-associated clinical disease and pulmonary distress, irrespective of the virus' antigenic subtype or susceptibility to antiviral medications, which will be paramount in limiting the devastating effects of current and future influenza outbreaks.

Thus, there is a need for novel methods and systems for achieving potent immune response to viral infections irrespective of the virus' antigenic subtype or susceptibility to antiviral medications.

### SUMMARY OF THE INVENTION

The present invention relates to the use of ultraviolet light for treating viral infections by stimulating cell mediated immunity (CMI) and other related immune responses with an intention to enhance the subject's immunity defense against replicating virus in the absence of antiretroviral agents. Indeed, the present invention is based in part on the discovery that extra-corporal irradiation of whole blood with pulsed-high energy ultraviolet (“UV”) light, followed by re-infusion of treated blood in to the subject, leads to activation of subject's CMI response. The Hemo-Modulator (“H-M”) device is used to irradiate the infected blood. An exemplary H-M device is disclosed in U.S. patent application Ser. No. 11/441,547, which is incorporated by reference herein.

According to some embodiments, the ultraviolet light (wavelength range 200 nm-400 nm) can be used to irradiate the virus and thus elicit cell-mediated immune response to fight the infection. Additionally, it has been discovered that during irradiation by the ultraviolet light, RNA interference (RNAi) can be introduced as one of the biological mechanisms to activate cell-mediated immunity.

In some embodiments, the invention relates to a method of treating a viral infection including applying ultraviolet light to a blood sample containing a viral particle and stimulating the subject's immune system to activate potent CMI against the virus. In some embodiments, the CMI activation further results in decreased viral load. In other embodiments, CMI activation further reduces cellular inflammation associated with active viral infection. In other embodiments, CMI activation further inhibits virus-induced inflammation.

In other embodiments, the CMI activation can be represented by cytokine activation. In other embodiment, the cytokine can be selected from a group consisting of IL1, IL2, IL3, IL4, IL5, IL6, IL7, IL8, IL9, IL10, IL11 and IL12.

In some embodiments, the virus causing an infection can be a Ribonucleic Acid (RNA) virus. In some embodiments, the RNA virus can be a virus selected from a group including Influenza and Human Immunodeficiency Virus (HIV).

In other embodiments, the invention relates to a method of treating HIV infection including applying ultraviolet light to a blood sample containing an HIV particle and thus stimulating the subject's immune system to activate potent CMI and other immune responses against the HIV virus.

In some other embodiments, the invention relates to a method of treating influenza infection, including applying ultraviolet light to a blood sample containing an influenza particle and stimulating the subject's immune system to activate potent CMI and other immune responses against the HIV virus

In further embodiments, CMI activation limits scope and severity of clinical disease associated with a response to the virus by a subject regardless of antigenic sub-type of the virus or susceptibility to anti-viral medication.

The methods of the present invention can use the ultraviolet light application wherein a wavelength of the ultraviolet light is in the range of 200 nm to 400 nm.

Unless otherwise defined, all technical and scientific terms used herein have the same meaning as commonly understood by one of ordinary skill in the art to which this invention relates. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

The present invention is described with reference to the accompanying drawings. In the drawings, like reference numbers indicate identical or functionally similar elements. Additionally, the left-most digit(s) of a reference number identifies the drawing in which the reference number first appears.

FIG. 1 is an exemplary plot illustrating effects between infected animals but not treated with H-M (SHAM animals) and animals treated in accordance with embodiments of the present invention.

FIG. 2 is an exemplary photograph illustrating clinical differences between SHAM-animals and animals treated with H-M. in accordance with embodiments of the present invention.

FIG. 3 is an exemplary plot illustrating effects on an ability to breathe by the SHAM-animals and animals treated in accordance with embodiments of the present invention.

FIG. 4 is an exemplary plot illustrating inflammatory responses by the SHAM-animals and animals treated in accordance with embodiments of the present invention.

FIG. 5 presents exemplary photographs of lung sections that were untreated and treated in accordance with embodiments of the present invention.

FIG. 6 presents exemplary photographs of lung sections showing cellular infiltration in animals that were untreated and treated in accordance with embodiments of the present invention.

FIG. 7 presents additional exemplary photographs of lung sections of animals that were untreated and treated in accordance with embodiments of the present invention.

FIG. 8 is a plot illustrating simian immunodeficiency (SIV) plasma virus loads post-treatment with an exemplary H-M for the AV89 Monkey.

FIG. 9 is plot illustrating 7/4 Fold increase in gag/env responses in the AV89 Monkey.

FIG. 10 is another plot illustrating SIV plasma virus loads post-treatment with an exemplary H-M for the T687 monkey.

FIG. 11 is another plot illustrating 4 Fold increase in gag response for the T687 Monkey.

FIG. 12 is yet another plot illustrating SIV plasma virus loads post-treatment with an exemplary H-M for the CN85 Monkey.

FIG. 13 is a plot illustrating immune response for the CN85 Monkey.

FIG. 14 is a plot illustrating SIV plasma viral loads in untreated Monkeys FIGS. 15A and 15B are plots illustrating cytokine induction pre and post H-M treatment in Rhesus Monkeys.

### DETAILED DESCRIPTION OF THE INVENTION

The present invention relates to an application of ultraviolet light (200 nm-400 nm) to treat viral infections by stimulating the subject's immune system to activate CMI and other relevant immune responses against the virus.

According to some embodiments, the ultraviolet light (wavelength range 200 nm-400 nm) can be used to inactivate the virus and stimulate the CMI and other related immune responses to fight the viral infection. Additionally, it has been discovered that during irradiation by the ultraviolet light, RNA interference (RNAi) may be introduced as one of the biological mechanisms to activate CMI.

In some embodiments, the invention relates to applying ultraviolet light to a blood sample containing a viral particle and thus stimulating the subject immune system to activate potent CMI and other immune responses against the virus The CMI can relate to an immune response that involves the activation of macrophages, natural killer cells (NK), antigen-specific cytotoxic T-lymphocytes and the release of various cytokines, such as for example IL I-IL 12, in response to an antigen.

Cells with cytotoxic activity contribute greatly to immune responses. An exemplary immune cell can be a cell of hematopoietic origin that is involved in the recognition of antigens. Immune cells include antigen presenting cells (APCs), such as dendritic cells or macrophages, B cells, T cells, neutrophils, natural killer (NK) cells, etc.

In the treatment of viral infections, an enhanced immune response can be beneficial and therefore, can be aided by increases in cytotoxic activity. For example, treatment of HIV infection can benefit from the ability to improve cytotoxic effects. Cytotoxic T lymphocytes (CTLs) can be implicated as essential but not sufficient to provide a robust immune response directed to HIV infection. (Addo et al. 2003 J. Virol. 77:2081.) HIV infection is thought to evade immune surveillance for various reasons including loss of T cells, viral mutational escape of HIV virions, and direct effects of HIV proteins. Improving CTL cytotoxic activity against HIV virions could potentially enhance the overall immune response against HIV infection.

Methods of the present invention can be beneficial with respect to treatment and/or management of viral infections, particularly for subjects with primary infection, those with chronic infection and those with any relevant opportunistic infections. The degree of immunological containment achieved by any given subject can be a function of their disease progression, history of the disease, prior viral treatment, genetic predisposition and/or other factors. In some embodiments of the present invention, application of the ultraviolet light results in CMI activation and thus further decrease in the subject's viral load. In other embodiments, the application of the viral load results in CMI activation which further reduces cellular inflammation associated with active viral infection. In other embodiments, the application of ultraviolet light results in CMI activation which further inhibits virus-induced inflammation associated with active viral infection.

Efficacy of the methods of the present invention and any adverse side effects can be monitored throughout the treatment of a subject using any of the methods available in the art, including those described in the examples below. A subject's vital signs, renal and liver function, glucose levels, etc., can be measured at predetermined time intervals. Blood samples can be analyzed for viral load using any protocol known to those skilled in the art.

Peripheral Blood Mononuclear Cells (PBMCS) can be collected from a subject at specific intervals, such as, for example, weekly or biweekly, and tested for viral load. These methods of detection can additionally be used to determine the presence of replicating HIV in lymph node samples obtained from a subject undergoing treatment in accordance with the methods of the invention. For example, the presence of replicating HIV in plasma can be determined using a branched chain DNA assay (bDNA), which has a lower limit of detection (LLD) of 50 HIV RNA molecules/ml (see Jacobson et al. (1996) Proc. Natl. Acad. Sci. USA 93:10405 10410; herein incorporated by reference). The presence of replicating HIV in lymph nodes can be determined using, for example, a co-culture assay (Chun (1999) Nature 401:874 875, herein incorporated by reference).

In some embodiments of the present invention, the viral infection can be caused by an RNA virus. As used herein the term “RNA virus” describes single stranded negative-sense and positive-sense RNA viruses. Positive-sense viral RNA is identical to viral mRNA and thus can be immediately translated by the subject cell. On the other hand, negative-sense viral RNA is complementary to mRNA and thus must be converted to positive-sense RNA by an RNA polymerase before translation.

In some embodiments, the RNA virus can be a virus selected from a group having Influenza and HIV. The HIV virus can be transmitted as single-stranded, positive-sense, enveloped virus. Upon entry of a target cell, the viral RNA genome can be converted to double-stranded DNA by a virally encoded reverse transcriptase that is present in the virus particle. Once the virus has infected the cell, two pathways are possible: either the virus becomes latent and the infected cell continues to function, or the virus can become active and replicate, and a large number of virus particles are liberated that can then infect other cells.

Two species of HIV can infect patients: HIV-1 and HIV-2. The HIV-1 virus can be more virulent and more easily transmitted. HIV-2 virus can weaken the immune system at a much slower rate as compared to HIV-1.

In some embodiments, the RNA virus can be an Influenza virus. The influenza virions include of an internal ribonucleoprotein core (a helical nucleocapsid) containing the single-stranded RNA genome, and an outer lipoprotein envelope lined inside by a matrix protein (H). Influenza virus can be categorized as Influenza A, B and C. Influenza B can be a single-stranded RNA virus which mostly infects humans and seals. In humans, influenza B mutates at rate 2-3 times lower than Influenza type A and lasting immunity may not possible for this virus. Influenza C can be a single-stranded RNA virus known to infect humans and pigs.

In some embodiments of the instant invention, the influenza virus can be an avian influenza A (H5N1). The H5N1 can be a subtype of the Influenza A virus which can cause illness in humans and many other animal species. The H5N1 can be the causative agent of “bird flu”.

In other embodiments, CMI activation can be represented by cytokine activation. Cytokines play a role in both innate and adaptive immune responses. Due to their central role in the immune system, cytokines may be involved in a variety of immunological, inflammatory and infectious diseases. In some embodiments, the cytokine can be selected from a group having IL1, IL2, IL3, IL4, IL5, IL6, IL7, IL8, IL9, IL10, IL11 and IL12.

The instant methods of treating a viral infection can be combined with any known antiviral treatments. In some embodiments, the instant methods of treating HIV infections can be combined with anti-retroviral agents, including: (1) nucleoside reverse transcriptase inhibitors, (2) non-nucleoside reverse transcriptase inhibitors, (3) protease inhibitors, (4) virus uptake/adsorption inhibitors, (5) virus receptor antagonists, (6) viral fusion inhibitors, (7) viral integrase inhibitors, and (8) transcription inhibitors, and the like.

In some embodiments, the anti-retroviral agents include reverse transcriptase inhibitors. In another embodiment, the inhibitors include nucleoside/nucleotide reverse transcriptase inhibitors, which are nucleoside or nucleotide analogs that inhibit action of the viral reverse transcriptase required for conversion of the viral RNA into deoxyribonucleic acid (DNA) during viral replication. These inhibitors include without limitation azidothymidine and its derivatives (e.g., AZT, Zidovudine), (2R,cis)-4-amino-1-(2-hydroxymethyl-1-1-oxathiolan-5-yl)-(1H)-pyrimidine-2-one (i.e., Lamivudine), 2′,3′-dideoxyinosine (didanosine), 2′,3′-dideoxycytidine (i.e., Zalcitabine), 2′,3′-didehydro-3′-deoxythymidine (i.e., stavudine), (1S,cis)-4-[2-amino-6-(cyclopropylamino)-9H-purin-9-yl]-2-cyclopentene-1-methanol sulfate (i.e., abacavir), (−)-beta-2′,3′-dideoxy-5-fluoro-3′-thiacytidine (i.e., emtricitabine), and phosphonate 9-R-(2-phosphonomethoxypropyl)adenine (i.e., PMPA; tenofovir disoproxil fumarate; adefovir) and various derivatives thereof (see for example, Deeks, S. G. et al., Antimicrob. Agents Chemother. 42(9):2380 2384 (1998). As provided by the examples, the nucleoside/nucleotide reverse transcriptase inhibitors are generally cyclic or acyclic nucleoside or nucleotide analogs.

In other embodiments, the antiviral agents include non-nucleoside reverse transcriptase inhibitors (NNRTI). These agents also inhibit the action of viral reverse transcriptase by binding to the enzyme and disrupting its catalytic activity. Inhibitors include, but are not limited to, 11-cyclopropyl-5,11-dihydro-4-methyl-6H-dipyrido-[3,3-b-2′,3′-][1,4]diaze-pin-6-one (i.e., Nevirapine); piperazine, 1-[3-[(1-methyl-ethyl)amino]-2-pyridinyl]-4-[[5-(methylsulfonyl)amino]-1-H-indol-2-yl]carbonyl]-, monomethane sulfonate (i.e., Delavirdine); and (S)-6-chloro-4-(cyclopropylethynyl)-1,4-dihydro-4-(trifluoromethyl)-2H-3,-1-benzoxazine-2-one (i.e., Efavirenz). Other include quinazolinone and it derivatives, for example trifluoromethyl-containing quinazolin-2(1H)-ones (Corbett, J. W. et al., Prog. Med. Chem. 40:63 105 (2000); calanolide A (Newman, R. A. et al. J. Pharm. Sci. 87(9):1077 1080 (1998); and 6-arylmethyl-1-(ethoxymethyl)-5-alkyluracil (i.e., emivirine) and its analogs (see El-Brollosy, N. R., J. Med. Chem. 45(26):5721 5726 (2002)).

In other embodiments, the antiviral agents include protease inhibitors. Without being bound by theory, protease inhibitors appear to inhibit HIV replication at the postintegrational level after the virus is integrated into the host chromosome. The target HIV protease enzyme, a 99-amino acid homodimer, cleaves pol-gag polypeptides on the viral envelope. The gag-pol precursor contains the amino acid sequences of various HIV proteins, such as proteins that form the capsid (p19) and nucleocapsid (p24). In addition, gag-pol also contains the sequence of retroviral enzymes, such as reverse transcriptase, proteases, and integrase. Inhibition of the HIV protease results in release of immature, noninfectious viral particles. Many of the protease inhibitors may also exert additional antiviral effects by inhibiting proteasome function in the cells. Protease inhibitors useful in the present invention include without limitation the agents indinavir, saquinavir (fortovase), ritonavir, nelfinavir, amprenavir, and lopinavir.

HIV virus replication may also be affected by inhibiting the action of integrase, a viral protein involved in inserting the human immunodeficiency virus type 1 (HIV-1) proviral DNA into the host genome. This class of inhibitors may include small molecule inhibitors or peptide inhibitors. Small molecule inhibitors, include, among others, integramycin (Singh, S. B. et al, Org. Lett. 4(7):1123 1126 (2002); (Vandegraaff, N. et al., Antimicrob. Agents Chemother. 45(9):2510 2516 (2001); polyhydroxylated styrylquinolines (Zouhiri, F. et al., J. Med. Chem. 43(8):1533 1540 (2000); and cyclodidemniserinol trisulfate (Mitchell, S. S. et al., Org. Lett. 2(11):1605 1607 (2000). Peptide based inhibitors include, among others, linear peptides (Puras Lutzke R. A. et al., Proc. Natl. Acad. Sci. USA 92(25):11456 11460 (1995); de Soultrait V. R. et al., J Mol. Biol. 318(1):45 58; cyclic peptides (Singh, S. B. et al., J. Nat. Prod. 64(7):874 882 (2001); and antibodies that bind and inhibit integrase activity (Yi, J. et al., J. Biol. Chem. 277(14):12164 12174 (2002). All references are hereby incorporated by reference.

In other embodiments, the instant methods of treating influenza infections can be combined with anti-retroviral agents, including Tamiflu (Oseltamivir). Tamiflu is the latest of the neuraminidase inhibitor (NI) class of medicines designed specifically to prevent the influenza virus from spreading and infecting other cells. It is effective against all common strains of influenza (types A and B). The medication targets one of two major surface structures on the influenza virus, the neuraminidase protein. The neuraminidase protein is virtually the same in all common strains of influenza. If neuraminidase is inhibited, the virus is not able to infect new cells.

In some embodiments, a microarray analysis can be performed to identify genes expressed as a results of H-M treatment. Microarray technology can be used as a tool for analyzing gene or protein expression, comprising a small membrane or solid support (such as but not limited to microscope glass slides, plastic supports, silicon chips or wafers with or without fiber optic detection means, and membranes including nitrocellulose, nylon, or polyvinylidene fluoride). The solid support can be chemically (such as silanes, streptavidin, and numerous other examples) or physically derivatized (for example, photolithography) to enable binding of the analyte of interest, usually nucleic acids, proteins, or metabolites or fragments thereof. The nucleic acid or protein can be printed (i.e., inkjet printing), spotted, or synthesized in situ. Deposition of the nucleic acid or protein of interest can be achieved by xyz robotic microarrayers, which utilize automated spotting devices with very precise movement controls on the x-, y-, and z-axes, in combination with pin technology to provide accurate, reproducible spots on the arrays. The analytes of interest are placed on the solid support in an orderly or fixed arrangement so as to facilitate easy identification of a particularly desired analyte. A number of microarray formats are commercially available from, inter alia, Affymetrix, ArrayIt, Agilent Technologies, Asper Biotech, BioMicro, CombiMatrix, GenePix, Nanogen, and Roche Diagnostics.

The nucleic acid or protein of interest can be synthesized in the presence of nucleotides or amino acids tagged with one or more detectable labels. Such labels include, for example, fluorescent dyes and chemiluminescent labels. In particular, for microarray detection, fluorescent dyes such as but not limited to rhodamine, fluorescein, phycoerythrin, cyanine dyes like Cy3 and Cy5, and conjugates like streptavidin-phycoerythrin (when nucleic acids or proteins are tagged with biotin) are frequently used. Detection of fluorescent signals and image acquisition are typically achieved using confocal fluorescence laser scanning or photomultiplier tube, which provide relative signal intensities and ratios of analyte abundance for the nucleic acids or proteins represented on the array. A wide variety of different scanning instruments are available, and a number of image acquisition and quantification packages are associated with them, which allow for numerical evaluation of combined selection criteria to define optimal scanning conditions, such as median value, inter-quartile range (IQR), count of saturated spots, and linear regression between pair-wise scans (r² and P). Reproducibility of the scans, as well as optimization of scanning conditions, background correction, and normalization, are assessed prior to data analysis.

Normalization refers to a collection of processes that are used to adjust data means or variances for effects resulting from systematic non-biological differences between arrays, subarrays (or print-tip groups), and dye-label channels. An array is defined as the entire set of target probes on the chip or solid support. A subarray or print-tip group refers to a subset of those target probes deposited by the same print-tip, which can be identified as distinct, smaller arrays of proves within the full array. The dye-label channel refers to the fluorescence frequency of the target sample hybridized to the chip. Experiments where two differently dye-labeled samples are mixed and hybridized to the same chip are referred to in the art as “dual-dye experiments”, which result in a relative, rather than absolute, expression value for each target on the array, often represented as the log of the ratio between “red” channel and “green channel.” Normalization can be performed according to ratiometric or absolute value methods. Ratiometric analyses are mainly employed in dual-dye experiments where one channel or array is considered in relation to a common reference. A ratio of expression for each target probe is calculated between test and reference sample, followed by a transformation of the ratio into log₂(ratio) to symmetrically represent relative changes. Absolute value methods are used frequently in single-dye experiments or dual-dye experiments where there is no suitable reference for a channel or array. Relevant “hits” are defined as expression levels or amounts that characterize a specific experimental condition. Usually, these are nucleic acids or proteins in which the expression levels differ significantly between different experimental conditions, usually by comparison of the expression levels of a nucleic acid or protein in the different conditions and analyzing the relative expression (“fold change”) of the nucleic acid or protein and the ratio of its expression level in one set of samples to its expression in another set.

Data obtained from microarray experiments can be analyzed by any one of numerous statistical analyses, such as clustering methods and scoring methods. Clustering methods attempt to identify targets (such as nucleic acids and/or proteins) that behave similarly across a range of conditions or samples. The motivation to find such targets is driven by the assumption that targets that demonstrate similar patterns of expression share common characteristics, such as common regulatory elements, common functions, or common cellular origins.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

### EXAMPLES

### Example 1

### Immunomodulation of Single Stranded RNA Virus, Such as Influenza

This example demonstrates the ability of the H-M device to modulate influenza-associated clinical disease and associated pulmonary distress, irrespective of the virus's antigenic subtype.

It has been established that clinical illness, general malaise, and respiratory distress that are present following an influenza infection are due in large part to the body's own response to the infection and not necessarily to the virus infection itself. The premise of the study described in this Example was that application of H-M device affects clinical illness and pulmonary disease through a possible immunomodulatory effects.

For these experiments, the more severe and devastating Spanish Flue, HINI (5000 TCID50), which is probably more clinically similar to a H₅N₁ challenge than a common influenza and a lower 500 TCID50 were administered to animals. The following groups of animals were evaluated through application of influenza dose and H-M treatment. (See, Table 1).

TABLE 1
Description of Groups and Treatment Protocols.

| Group Name | Influenza Dose | H-M Treatment |
|---|---|---|
| Sham | 0 | No |
| ShamP | 0 | Yes |
| 500 | 500 | No |
| 500P | 500 | Yes |
| 5000 | 5000 | No |
| 5000P | 5000 | Yes |

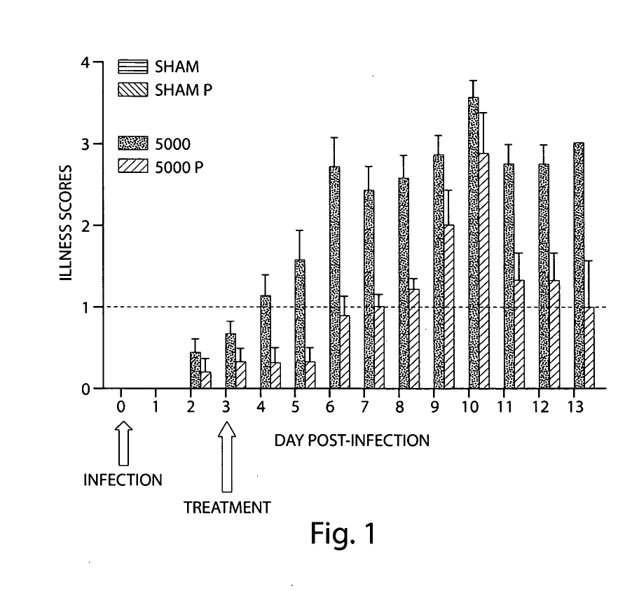

As summarized in Table 1, some subjects were not infected while others were infected with various dosages of viruses. Further, infected and non infected subjects received H-M treatment in accordance with systems and methods of the present invention. Specifically, animals infected with 5000 TCID50 of influenza and treated once by H-M 3 days post-infection (5000p) showed substantial clinical improvement over untreated controls. Specifically, minimal clinical illness was observed up to 9 days post infection; whereas the SHAM (5000) developed severe clinical illness by day 6 (See, FIG. 1). For these experiments, a group of non infected subjects which was not treated by the H-M treatment was used as a control.

Further, both treated and untreated infected groups showed substantial clinical illness at days 9 and 10. However, the observed clinical illness at these points appeared to be for different reasons. The treated group (5000p) appeared to suffer from overall dehydration and weight loss, a common side effect of influenza infection, while maintaining signs of activity and overall awareness. In contrast, the untreated infected group appeared to be suffering from a combination of effects that included an overall malaise, lack of mobility, and an unawareness of surroundings, in addition to severe dehydration and weight loss. These distinctions are important, since clinically, dehydration and nourishment would be easily treated in a hospital setting.

Further, as shown in FIG. 1, by day 11 the treated group had recovered to a mild clinical illness; whereas, the untreated group still exhibited severe clinical illness. Thus, effects of H-M treatment were particularly and obviously visible between the treated and untreated animals at later times.

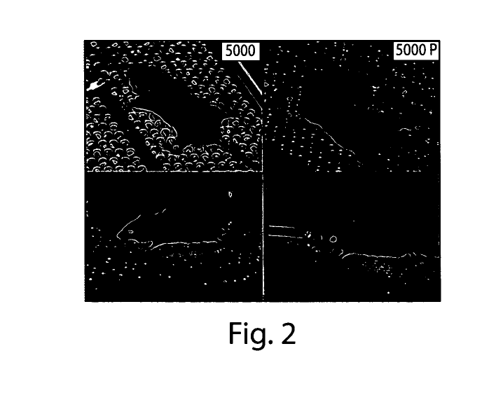

Representative photographs of animals 13 days post infection illustrate readily observable clinical differences between these two groups (See, FIG. 2). Animals that were treated by H-M, (5000P) were bright-eyed, had kept fur, were very active and alert, and only showed minor signs of dehydration (See, FIG. 2). In contrast, SHAM (5000) animals exhibited no activity, total lethargy, hunched posture, a ruffled coat, severe dehydration, and squinted/closed eyes.

In summary, a substantial reduction in clinical illness was observed in the H-M treated group.

Pulmonary/Respiratory Disease

A. Lung Airway Function

Hallmarks of influenza infections are a severe respiratory disease and an overall inability to breathe. Thus, the relative respiratory function of infected animals was determined by measuring airway resistance. The greater the airway resistance in these studies, the more difficult breathing is for these animals. As can be understood by one skilled in the art, the present invention is designed to treat severe respiratory disease associated with any viral and non-viral infections in animals, humans, or other subjects. For illustrative purposes only, the following description will refer to treatment of influenza (caused by injection of TCID50) in animals.

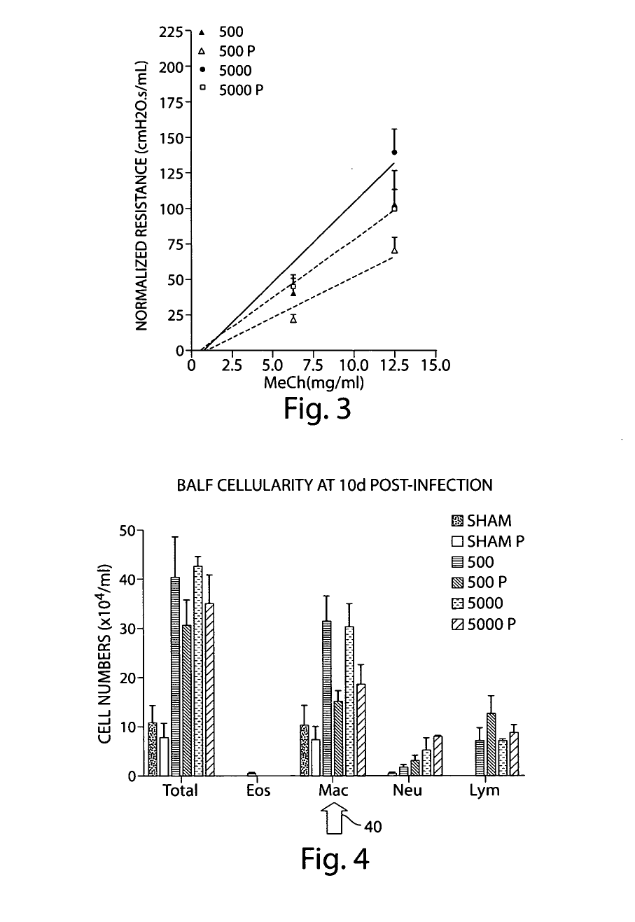

As shown in FIG. 3, both the 500 TCID50 (500P) and 5000 TCID50 (5000P) infected groups that were treated by H-M exhibited a significantly greater ability to breathe relative to their SHAM counterparts (500 and 5000, respectively). In fact, the 5000P group had an identical airway resistance to the 500 TCID50 group, which showed only very minimal clinical signs throughout the study.

These results unequivocally demonstrate that despite an active viral infection at a severe infectious dose, treatment by H-M appeared to greatly enhance airway function in the 5000P group.

B. Inflammation in the Lung

Decreased lung function and inability to breathe is primarily due to infiltration of inflammatory cells into the lung causing severe pneumonia.

Isolation and identification of cells within the conducting airways of the lung air is a good indication of the inflammatory events taking place that ultimately restricts the ability of an animal to breathe. Following infection with influenza viruses, the lungs of both treated and untreated animals display an increase in the total amount of inflammatory cells present (See, FIG. 4). However, animals treated by H-M exhibit a trend towards fewer cell numbers and thus less inflammation in the lung tissue.

At day 10 post infection, the animals that were treated by H-M (500p and 5000p) had significantly fewer macrophages (green arrow 40, FIG. 4), indicating that the inflammatory response at this time point had been significantly reduced.

C. Overall Lung Pathology

In normal undamaged lungs, there should be clearly open airways, little to no cellular infiltration or blood in these spaces, no indications of cellular destruction, and an overall open honeycomb appearance. These open spaces permit a free exchange of oxygen within the lung and are therefore critical to its function. Pathological examination of lungs clearly indicated that H-M treatment significantly inhibited virus-induced inflammation resulting in airways that were clear of inflammatory cells and cellular debris.

1. Sham Infected Animals

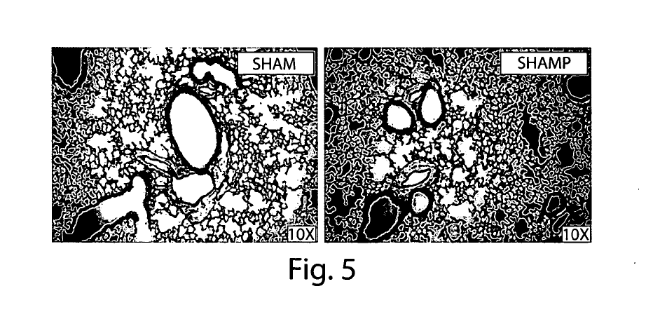

Pre-pathological examination of lung sections from animals that were either treated or untreated by H-M showed an open architecture of a normal well functioning lung (See, FIG. 5). Thus, it is a clear indication that treatment by H-M did not produce any readily observable adverse affects.

II. 5000 TCID50 Infected Animals

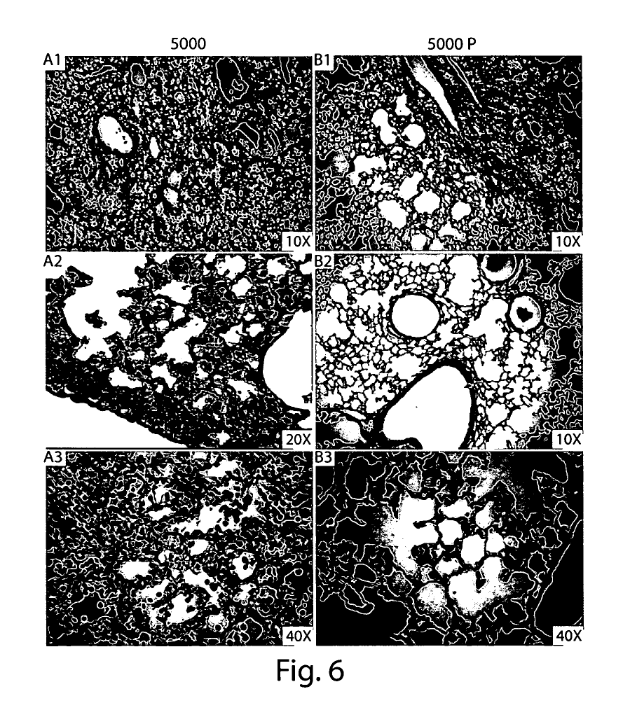

The 5000P group did show foci of cellular infiltration within the lung (See, FIG. 6, quadrant B1) that were fairly confined (See, arrows 62 and 64 in quadrant B1 that indicate sites of cellular infiltration (red arrows 62) and sites of open airways (green arrow 64)).

Overall, the lungs of the 5000P treated group showed the open airway architecture with little to no damage in the cell lining of the larger airways and only confined areas of inflammation (See, FIG. 6, quadrants B2 and B3).

In contrast, the SHAM 5000 group showed inflammation across large areas of the lung (See, FIG. 6, quadrants A1 and A3). In addition to the areas of infiltration, blood cells were readily observable within the airways (See, FIG. 6, quadrant A2) indicating that severe damage of the lung had occurred.

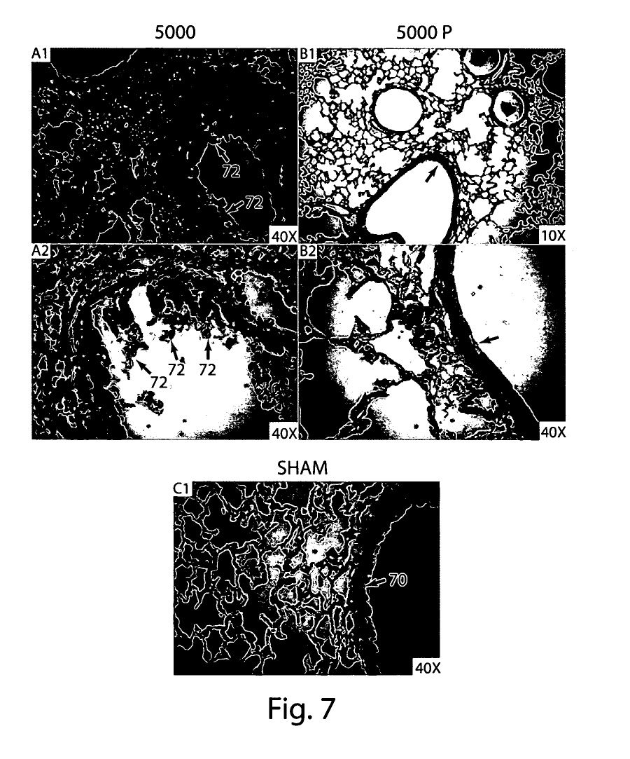

The columnar shaped cells lining the larger airways of 5000 P H-M treated were largely intact (See, FIG. 7 quadrants B1 and B2). Arrows 70 indicate intact cells lining the airways.

In contrast, the cells lining the large airways of 5000 SHAM animals were either destroyed or sloughing off into the airways (See, FIG. 7, quadrants A1 and A2). Red arrows 72 indicate sites of cell destruction and loss of airway lining. In addition, hemorrhaging, as indicated by the presence of red blood cells near the passageways, was readily observed (See, FIG. 7, quadrant A1).

Thus, H-M treatment significantly inhibited virus-induced inflammation and thus improved the breathing ability of the treated animals.

### Example 2

### Immunomodulation of a Single Stranded RNA Virus Such as HIV

Immune Stimulation and Reduction in SIVmac Plasma Virus Load by Irradiation of Blood with Pulsed-High Energy Ultraviolet Light.

Cell Mediated Immunity (CMI) is a key component in suppressing SIV infections of Rhesus macaques (RhM). Potent cell mediated immune responses are associated with control of the plasma virus load (PVL). The hypothesis tested is that extracorporeal UV-irradiation of whole blood and its re-infusion would stimulate CMI and suppress PVL in SIV mac infected rhesus macaques. The H-M is used to irradiate blood.

Preliminary In Vitro Study: Before committing monkeys to in vivo studies, a preliminary in vitro study is carried out. For these experiments, the viral load is measured in untreated sample of SIV infected tissue culture [clear] fluids and used as a control for the subsequent experiments. The H-M system is used to irradiate the blood. The results of these experiments are summarized in Table 2. Specifically, the 2nd H-M treatment further reduces the live virus titer by 50% to 1536 TCID50, the 3rd H-M treatment further reduces the titer an additional 87.5% from 1536 TCID50 to 192 TCID50. In summary, the overall reduction in live virus titer is >99.9%. Thus, the procedure is deemed appropriate for testing in vivo.

TABLE 2
UV Inactivation of SIV Infected Tissue
Culture Fluids using the H-M

| Number of | |
|---|---|
| Treatments | TCID50 |
| Untreated | 45708.8 |
| One | 3072 |
| Two | 1536 |
| Three | 192 |

Extracorporeal Inactivation of Simian Immunodeficiency Virus in Blood and Immune Stimulation Using the H-M In Vivo.

For these experiments, the blood of infected monkeys is treated in the H-M to inactivate Simian Immunodeficiency Virus (SIV) and stimulate anti-viral immunity. This newly stimulated immunity could then act against SIV-infected white blood cells and thereby decrease the level of virus in the blood (called virus load). Virus load in the blood is the key factor in predicting the onset of AIDS. Monkeys (and HIV infected persons) with high virus loads develop AIDS more quickly than monkeys (or humans) with lower virus loads.

SIV is the simian counterpart to HIV. SIV causes AIDS in rhesus monkeys (Rhs). SIV infected Rhs are ideal for testing new immunotherapy because Rhs are primates and therefore closely related to humans. SIV infects the same types of white blood cells as HIV, making it an excellent AIDS model. SIV is a highly potent virus in Rh monkeys. Infected monkeys lose T cells as seen in humans and develop the same opportunistic infections such as Pneumocystis and atypical tuberculosis. Any promising results in this highly pathogenic AIDS model would strongly support clinical studies in HIV infected persons.

Preliminary studies described above unequivocally show that tissue culture grown SIV was 99.9% killed by H-M treatments. This result is important because it showed that H-M treatment directly kills the virus. For studies in the infected monkey, 3.3 ml/kg of whole blood (+anti-coagulant) from three SIV infected monkeys was UV-treated by passage through the H-M. Blood was re-infused immediately after treatment. Further, blood samples were taken for bDNA viral load test, ELISPOT and CD4/CD8+ cell number counts. Other routine blood values were also counted. The bDNA measurements were done as described in J. Clin. Microbiol. 1999, March 37(3) and ELISPOT measurements were done as described in F. Fujihashi et al., “Cytokine-Specific ELISPOT Assay, “J. Immunol. Meth. 160:181-189 (1993). Additional blood samples were taken to measure virus load and immune stimulation.

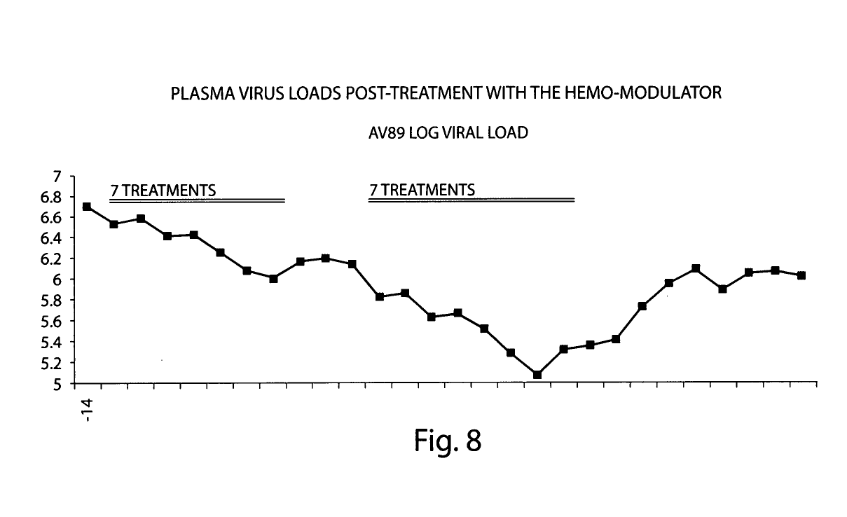

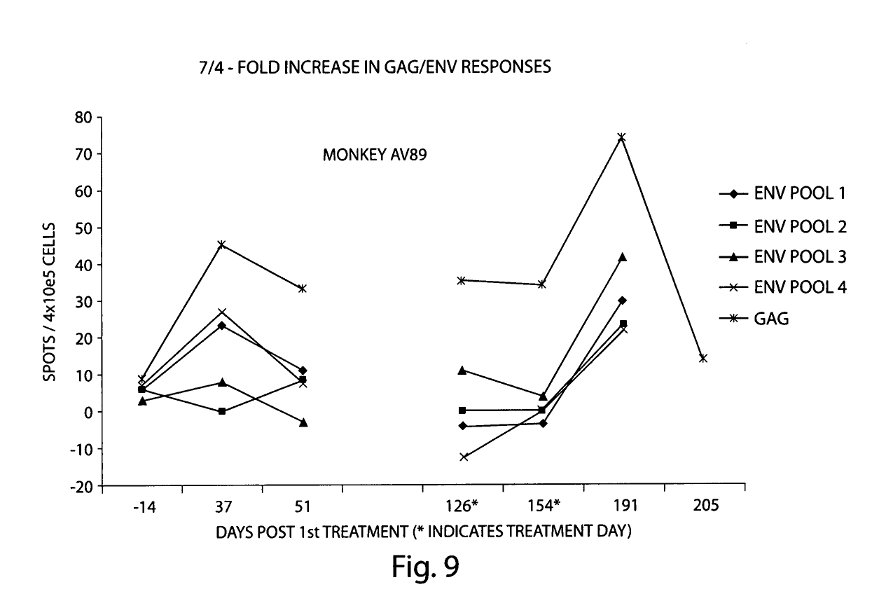

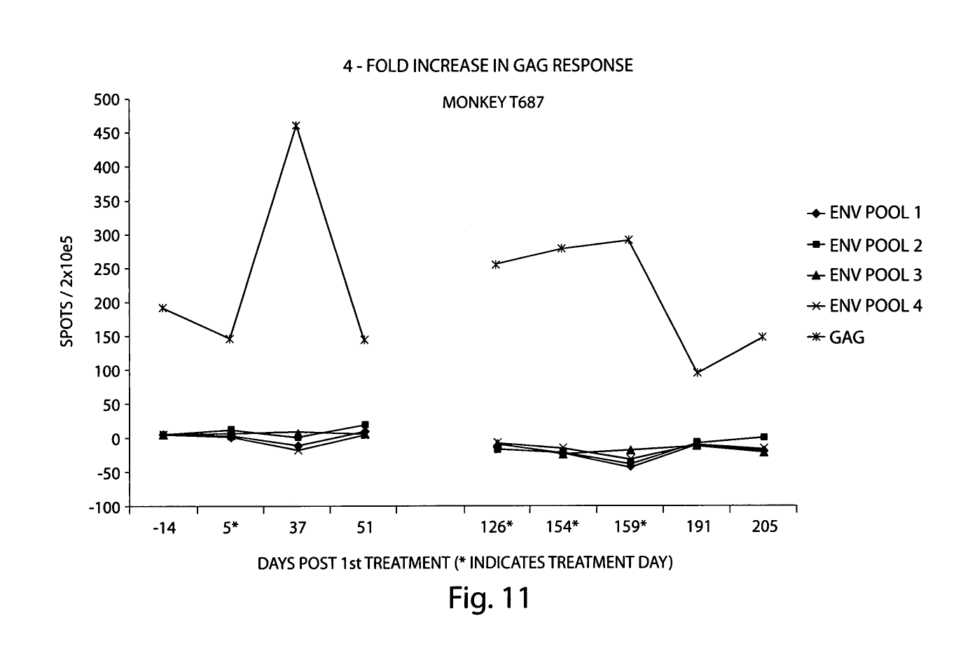

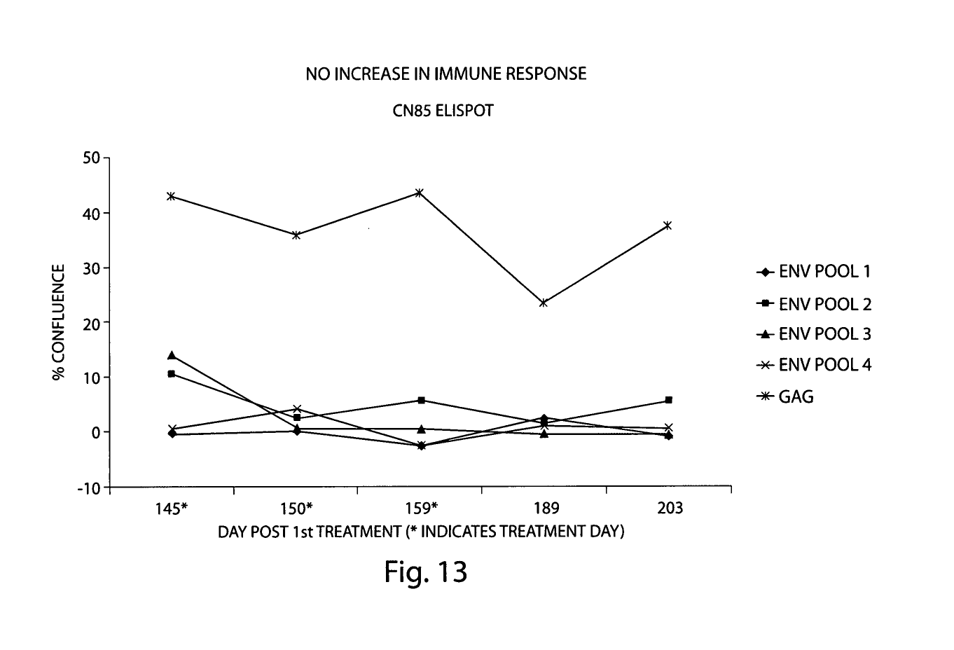

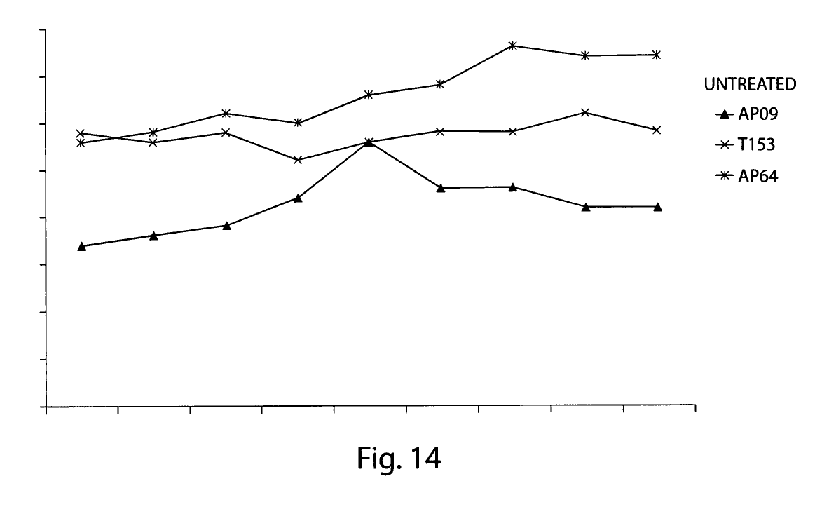

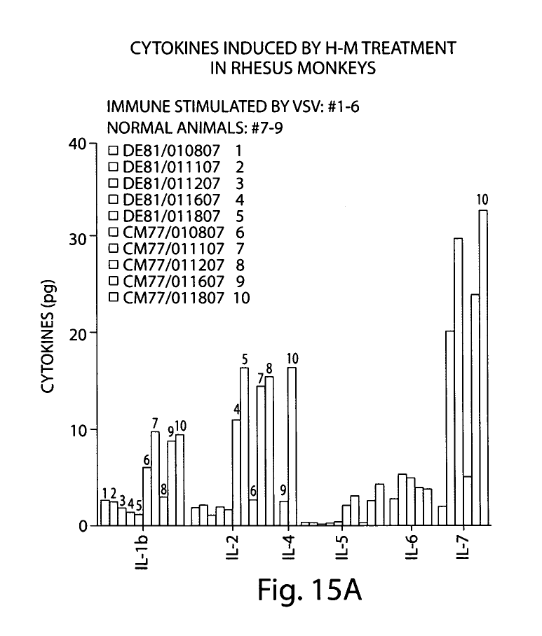

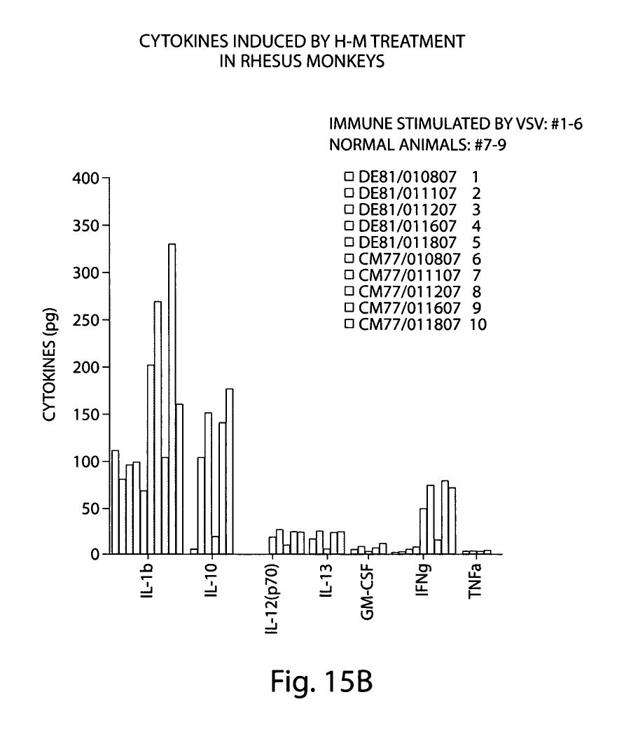

The results of these experiments are summarized in Table 3 and FIGS. 8-15. Specifically, FIG. 14 summarizes SIV plasma viral loads in untreated monkeys. In comparison, FIGS. 8-13 unequivocally show a significant decrease in viral load post-H-M treatment of the SIV infected monkeys. The results show that anti-viral immunity was significantly stimulated in of two of three monkeys.

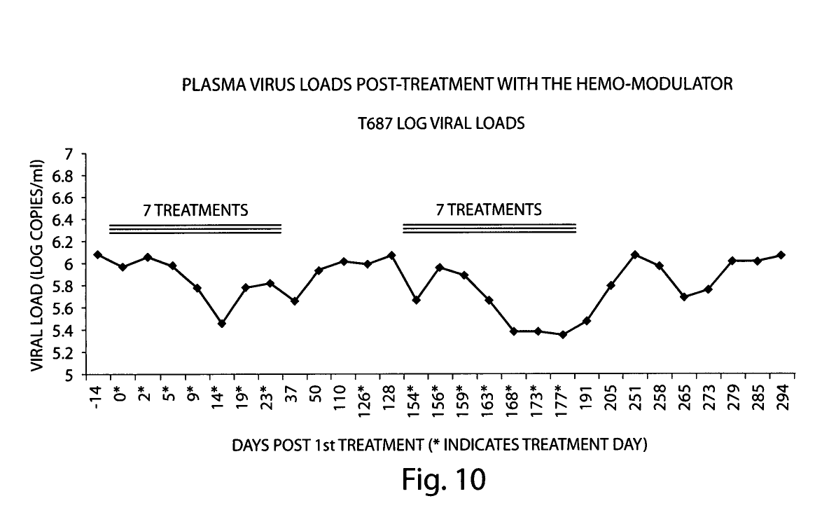

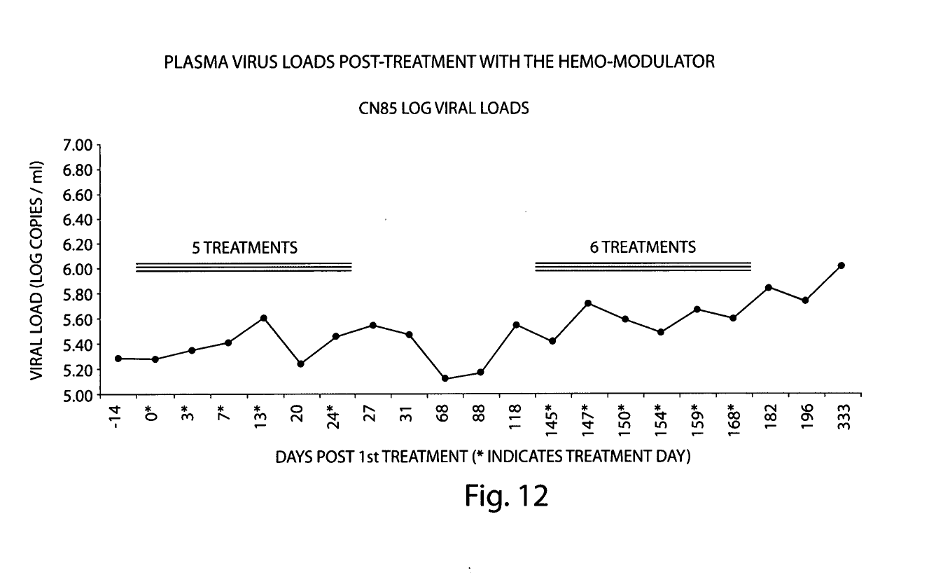

TABLE 3
SIVmac Infected Rhesus Monkeys - Whole
Blood UV Light Irradiation in the H-M
SIVmac Infected Rhesus Monkeys - Whole
Blood UV Light Irradiation in the H-M

| Rhesus | | | Pre-Treatment | Post-Treatment |
|---|---|---|---|---|
| Monkey | | Duration of | Plasma Virus | Plasma Virus |
| Number | Sex/Age | Infection | Load | Load |
| T687* | F/11 yrs | SIVmac239 - | 6.1 × 106 | See FIG. 10 |
| | | 2 yrs | | |
| CN85 | M/11 yrs | SIVmac251 - | 5.3 × 105 | See FIG. 12 |
| | | 16 mo | | |
| AV89 | F/13 yrs | SIVmac239 - | 6.7 × 106 | See FIG. 8 |
| | | 2 yrs | | |

*Mamu ao1+

The results of these studies are very significant. The positive anti-viral effects are directly linked to a stimulation of CMI. A possible mechanism may involve inactivation of SIV in the blood followed by uptake of the damaged virus by white blood cells called antigen processing cells (“APCs”). Stimulated APCs may then interact with T-cells capable of stimulating CMI. Further, as a result of application of H-M application cellular immunity is activated to Produce Cytotoxic T Killer Cells, Interferons or other anti-viral substances.

Further experiments have proved that H-M treatment leads to activation of immunity associated gene expression. The results of these experiments are summarized in FIGS. 15A and 15B and Table 4.

As summarized by FIGS. 15A and 15 B, H-M treatment results in activation of different cytokines such as IL1b, IL-2, IL-4, IL-5, IL-6, IL-7, IL-10, IL-12, IL13. Further, Table 4 summarizes gene array profile of various immunity associated genes which expression is altered as a result of H-M Treatment.

TABLE 4

| | | Fold Change |
|---|---|---|
| | Gene | (CM77_timepoint_{—} |
| Gene Title | Symbol | 24 h_baseline) |
| ADP-ribosylation factor-like 1 | ARL1 | −1.050238322 |
| ADP-ribosylation factor-like 11 | ARL11 | −1.104890226 |
| ADP-ribosylation-like factor 6 interacting protein 6 | ARL6IP6 | −1.159688997 |
| ADP-ribosylation-like factor 6 interacting protein 6 | ARL6IP6 | −1.089761734 |
| B and T lymphocyte associated | BTLA | −1.034379595 |
| B-cell receptor-associated protein 29 | BCAP29 | −1.025094695 |
| caspase 1, apoptosis-related cysteine peptidase (interleukin 1, beta, | CASP1 | −1.516726016 |
| convertase) | | |
| caspase 1, apoptosis-related cysteine peptidase (interleukin 1, beta, | CASP1 | −1.236206567 |
| convertase) | | |
| catenin (cadherin-associated protein), alpha 1, 102 kDa | CTNNA1 | −1.185963493 |
| CCAAT/enhancer binding protein (C/EBP), delta | CEBPD | −2.350357597 |
| CD163 molecule | CD163 | −1.130346481 |
| CD180 molecule | CD180 | −1.252345395 |
| CD200 receptor 1 | CD200R1 | −1.425514836 |
| CD200 receptor 1 | CD200R1 | −1.489003578 |
| CD276 molecule | CD276 | −1.819990361 |
| CD300 molecule-like family member b | CD300LB | −1.236478755 |
| CD300a molecule | CD300A | −1.132680469 |
| CD36 molecule (thrombospondin receptor) | CD36 | −2.471349974 |
| CD36 molecule (thrombospondin receptor) | CD36 | −2.031452048 |
| CD58 molecule | CD58 | −1.8640987 |
| CD58 molecule | CD58 | −2.071228425 |
| CD58 molecule | CD58 | −2.059798037 |
| CD58 molecule /// CD58 molecule | CD58 | −1.951651788 |
| CD74 molecule, major histocompatibility complex, class II invariant chain | CD74 | −1.244660924 |
| CDC42 effector protein (Rho GTPase binding) 3 | CDC42EP3 | −1.551881386 |
| CDK5 regulatory subunit associated protein 1-like 1 | CDKAL1 | −1.210697176 |
| Complement factor H-related 1 | CFHR1 | −1.194801916 |
| DEAD (Asp-Glu-Ala-Asp) box polypeptide 4 | DDX4 | −1.758308225 |
| DEAD/H (Asp-Glu-Ala-Asp/His) box polypeptide 11 (CHL1-like helicase | DDX11 /// | −1.437743418 |
| homolog, S. cerevisiae) /// DEAD/H (Asp-Glu-Ala-Asp/His) box polypeptide | DDX12 /// | |
| 12 (CHL1-like helicase homolog, S. cerevisiae) /// DEAD/H (Asp-Glu-Ala- | LOC642846 | |
| Asp/His) box polypeptide 11-like | | |
| epidermal growth factor receptor pathway substrate 15 | EPS15 | −1.040378051 |
| epidermal growth factor receptor pathway substrate 8 | EPS8 | −1.168711097 |
| immediate early response 2 | IER2 | −1.074083981 |
| immunoglobulin superfamily, member 4D | IGSF4D | −1.282735907 |
| interferon regulatory factor 5 | IRF5 | −1.233940625 |
| interferon, gamma-inducible protein 16 | IFI16 | −1.075046424 |
| interferon, kappa | IFNK | −1.294747218 |
| interleukin 1 family, member 6 (epsilon) | IL1F6 | −1.009155537 |
| interleukin 15 | IL15 | −1.253755921 |
| interleukin 16 (lymphocyte chemoattractant factor) | IL16 | −2.059362043 |
| jun oncogene | JUN | −1.377356317 |
| jun oncogene | JUN | −1.069024419 |
| killer cell lectin-like receptor subfamily D, member 1 | KLRD1 | −1.188966829 |
| major histocompatibility complex, class II, DQ beta 1 | HLA-DQB1 | −2.00471499 |
| Similar to bovine IgA regulatory protein | LOC492311 | −1.000851008 |
| SUMO1 activating enzyme subunit 1 | SAE1 | −1.569862203 |
| SUMO1/sentrin specific peptidase 1 | SENP1 | −1.193076652 |
| Suppression of tumorigenicity 18 (breast carcinoma) (zinc finger protein) | ST18 | −1.218484618 |
| suppressor of cytokine signaling 3 | SOCS3 | −1.024150876 |
| suppressor of cytokine signaling 3 | SOCS3 | −1.22862783 |
| T cell receptor associated transmembrane adaptor 1 | TRAT1 | −1.043868857 |
| T cell receptor gamma variable 5 /// | TRGV5 /// | −1.6101765 |
| hypothetical protein LOC648852 | LOC648852 | |
| TNF receptor-associated protein 1 | TRAP1 | −1.469560709 |
| TNFAIP3 interacting protein 2 | TNIP2 | −1.023186615 |
| toll-like receptor 8 | TLR8 | −1.6255767 |
| TRAF3 interacting protein 3 | TRAF3IP3 | −1.37283733 |
| transforming growth factor, beta-induced, 68 kDa | TGFBI | −1.02791421 |
| tumor necrosis factor (ligand) superfamily, | TNFSF13 /// | −1.138391069 |
| member 13 /// tumor necrosis factor (ligand) superfamily, | TNFSF12- | |
| member 12-member 13 | TNFSF13 | |
| tumor necrosis factor (ligand) superfamily, | TNFSF13 /// | −1.199911211 |
| member 13 /// tumor necrosis factor (ligand) superfamily, | TNFSF12- | |
| member 12-member 13 | TNFSF13 | |
| tumor necrosis factor receptor superfamily, member 10a | TNFRSF10A | −1.008302154 |
| tumor necrosis factor receptor superfamily, member 17 | TNFRSF17 | −1.789070055 |
| tumor necrosis factor, alpha-induced protein 6 | TNFAIP6 | −1.026443004 |
| tumor necrosis factor, alpha-induced protein 8 | TNFAIP8 | −1.47563284 |
| tumor necrosis factor, alpha-induced protein 8 | TNFAIP8 | −1.416455853 |
| tumor necrosis factor, alpha-induced protein 8-like 2 | TNFAIP8L2 | −1.512409326 |
| v-fos FBJ murine osteosarcoma viral oncogene homolog | FOS | −3.06618811 |
| Activated leukocyte cell adhesion molecule | ALCAM | 1.595913733 |
| Activated leukocyte cell adhesion molecule | ALCAM | 1.590731716 |
| B-cell CLL/lymphoma 11A (zinc finger protein) | BCL11A | 2.119797668 |
| B-cell CLL/lymphoma 2 | BCL2 | 1.255425326 |
| B-cell CLL/lymphoma 2 | BCL2 | 1.030092552 |
| B-cell CLL/lymphoma 9 | BCL9 | 3.036286028 |
| BCL2/adenovirus E1B 19 kDa interacting protein 3 | BNIP3 | 2.27423485 |
| BCL2-associated athanogene /// BCL2-associated athanogene | BAG1 | 1.056105026 |
| BCL2-associated transcription factor 1 | BCLAF1 | 2.77427347 |
| BCL2-like 11 (apoptosis facilitator) | BCL2L11 | 1.023621737 |
| BCL2-related protein A1 | BCL2A1 | 2.048848628 |
| BCL6 co-repressor | BCOR | 2.149873561 |
| BCL6 co-repressor-like 1 | BCORL1 | 1.552494446 |
| BCL6 co-repressor-like 1 | BCORL1 | 1.413641421 |
| Burkitt lymphoma receptor 1, GTP binding protein (chemokine (C-X-C motif) | BLR1 | 1.627776634 |
| receptor 5) | | |
| C1q and tumor necrosis factor related protein 3 /// C1q and tumor necrosis | C1QTNF3 | 1.966957503 |
| factor related protein 3 | | |
| C1q and tumor necrosis factor related protein 4 | C1QTNF4 | 2.11551907 |
| C1q and tumor necrosis factor related protein 9 | C1QTNF9 | 1.300962813 |
| CASP8 associated protein 2 | CASP8AP2 | 1.410371523 |
| CD24 molecule | CD24 | 1.405873786 |
| CD44 molecule (Indian blood group) | CD44 | 1.053654345 |
| CD5 molecule | CD5 | 1.438962762 |
| CD53 molecule | CD53 | 1.112098174 |
| CD55 molecule, decay accelerating factor for complement (Cromer blood | CD55 | 1.635392328 |
| group) | | |
| CD6 molecule | CD6 | 1.426788678 |
| CD6 molecule | CD6 | 1.13119078 |
| CD6 molecule | CD6 | 1.01263324 |
| CD83 molecule | CD83 | 2.244247837 |
| CD96 molecule | CD96 | 1.311962554 |
| CDC-like kinase 4 | CLK4 | 2.622947895 |
| CDK5 regulatory subunit associated protein 2 | CDK5RAP2 | 1.466474516 |
| checkpoint suppressor 1 | CHES1 | 1.180426389 |
| checkpoint suppressor 1 | CHES1 | 1.298241171 |
| chemokine (C-C motif) ligand 18 (pulmonary and activation-regulated) | CCL18 | 1.169874984 |
| chemokine (C-X-C motif) ligand 5 | CXCL5 | 2.365792257 |
| Chemokine-like factor | CKLF | 1.177324255 |
| colony stimulating factor 1 (macrophage) | CSF1 | 1.23620218 |
| complement component (3b/4b) receptor 1 (Knops blood group) | CR1 | 1.264004692 |
| Component of oligomeric golgi complex 2 | COG2 | 1.130095271 |
| CXXC finger 5 | CXXC5 | 2.513905365 |
| CXXC finger 5 | CXXC5 | 1.227042081 |
| CXXC finger 5 /// CXXC finger 5 | CXXC5 | 1.129077627 |
| Cyclin C | CCNC | 1.129094399 |
| cyclin F | CCNF | 3.556717123 |
| Cyclin I | CCNI | 1.036765164 |
| Cyclin I | CCNI | 2.204447147 |
| cyclin J | CCNJ | 2.539955168 |
| cyclin J-like | CCNJL | 1.473768369 |
| cyclin-dependent kinase 5, regulatory subunit 1 (p35) | CDK5R1 | 1.15616803 |
| cyclin-dependent kinase 6 | CDK6 | 1.53400165 |
| DEAD (Asp-Glu-Ala-Asp) box polypeptide 31 | DDX31 | 1.236635237 |
| DEAD (Asp-Glu-Ala-Asp) box polypeptide 50 | DDX50 | 1.373620446 |
| DEAD (Asp-Glu-Ala-Asp) box polypeptide 52 | DDX52 | 2.424825885 |
| DEAD (Asp-Glu-Ala-Asp) box polypeptide 54 /// DEAD (Asp-Glu-Ala-Asp) | DDX54 | 1.721274624 |
| box polypeptide 54 | | |
| DEAD (Asp-Glu-Ala-Asp) box polypeptide 55 | DDX55 | 1.430282242 |
| defensin, alpha 1 /// defensin, alpha 3, neutrophil-specific /// similar to | DEFA1 /// | 2.45659796 |
| Neutrophil defensin 1 precursor (HNP-1) (HP-1) (HP1) (Defensin, alpha 1) | DEFA3 /// | |
| | LOC728358 | |
| defensin, beta 126 /// defensin, beta 126 | DEFB126 | 2.371694976 |
| defensin, theta 1 pseudogene | DEFT1P | 3.805556291 |
| DNA-damage-inducible transcript 3 | DDIT3 | 2.114540909 |
| Epidermal growth factor receptor pathway substrate 15-like 1 | EPS15L1 | 1.811549922 |
| FAT tumor suppressor homolog 3 (Drosophila) | FAT3 | 1.055846392 |
| FAT tumor suppressor homolog 3 (Drosophila) | FAT3 | 1.236247722 |
| Fibroblast growth factor 12 | FGF12 | 1.559812983 |
| fibroblast growth factor 12 | FGF12 | 3.184680994 |
| Fibroblast growth factor 2 (basic) | FGF2 | 2.468424869 |
| fibroblast growth factor 5 | FGF5 | 3.190998854 |
| fibroblast growth factor 7 (keratinocyte growth factor) | FGF7 | 1.510968365 |
| fibroblast growth factor receptor 1 (fms-related tyrosine kinase 2, Pfeiffer | FGFR1 | 1.218978271 |
| syndrome) | | |
| fibroblast growth factor receptor substrate 2 | FRS2 | 1.006937593 |
| forkhead box L1 | FOXL1 | 2.455914701 |
| Forkhead box N1 | FOXN1 | 2.122955101 |
| Forkhead box O1A (rhabdomyosarcoma) | FOXO1A | 1.262142805 |
| Forkhead box O3A | FOXO3A | 1.722725982 |
| Forkhead box O3A | FOXO3A | 1.349241077 |
| forkhead box O3A | FOXO3A | 1.338975711 |
| Forkhead box P1 | FOXP1 | 3.221709698 |
| forkhead box P1 | FOXP1 | 1.077112851 |
| Forkhead box P1 | FOXP1 | 1.115396219 |
| FOS-like antigen 2 | FOSL2 | 1.045704886 |
| growth arrest and DNA-damage-inducible, beta | GADD45B | 1.08330297 |
| growth arrest and DNA-damage-inducible, beta | GADD45B | 1.280131439 |
| growth differentiation factor 9 | GDF9 | 1.799122828 |
| Hypoxia-inducible factor 1, alpha subunit (basic helix-loop-helix transcription | HIF1A | 1.422258761 |
| factor) | | |
| immediate early response 5 | IER5 | 1.319253046 |
| immediate early response 5-like | IER5L | 1.12543663 |
| Immunoglobulin heavy constant alpha 1 | IGHA1 | 2.785889354 |
| immunoglobulin heavy constant delta | IGHD | 1.599630135 |
| immunoglobulin heavy constant gamma 1 (G1m marker) | IGHG1 | 1.195087636 |
| Immunoglobulin heavy constant gamma 1 (G1m marker) /// Immunoglobulin | IGHG1 | 1.058202973 |
| heavy constant gamma 1 (G1m marker) | | |
| immunoglobulin superfamily, member 4 | IGSF4 | 1.938588988 |
| immunoglobulin superfamily, member 4D | IGSF4D | 3.611098468 |
| Interferon (alpha, beta and omega) receptor 2 | IFNAR2 | 1.343561007 |
| interferon regulatory factor 2 binding protein 2 | IRF2BP2 | 1.208430928 |
| interferon regulatory factor 4 | IRF4 | 3.678370748 |
| interferon-induced protein with tetratricopeptide repeats 2 | IFIT2 | 1.310249758 |
| interferon-induced protein with tetratricopeptide repeats 3 | IFIT3 | 1.571369147 |
| interleukin 1 receptor accessory protein | IL1RAP | 1.644464495 |
| interleukin 1 receptor antagonist | IL1RN | 1.978098988 |
| interleukin 1 receptor antagonist | IL1RN | 2.031082152 |
| interleukin 1 receptor, type I | IL1R1 | 1.975867566 |
| interleukin 1 receptor, type I | IL1R1 | 1.079724055 |
| interleukin 1 receptor, type II | IL1R2 | 1.386969109 |
| interleukin 1 receptor, type II | IL1R2 | 3.25443797 |
| interleukin 13 receptor, alpha 2 | IL13RA2 | 2.080890278 |
| Interleukin 17 receptor B | IL17RB | 1.018975144 |
| interleukin 18 binding protein | IL18BP | 1.635683595 |
| Interleukin 28 receptor, alpha (interferon, lambda receptor) | IL28RA | 3.609839015 |
| Interleukin 4 receptor | IL4R | 1.881536578 |
| interleukin 8 | IL8 | 2.532882388 |
| interleukin 9 receptor /// similar to Interleukin-9 receptor precursor (IL-9R) | IL9R /// | 1.834221056 |
| (CD129 antigen) | LOC729486 | |
| interleukin 9 receptor /// similar to Interleukin-9 receptor precursor (IL-9R) | IL9R /// | 1.266393454 |
| (CD129 antigen) | LOC729486 | |
| interleukin enhancer binding factor 3, 90 kDa | ILF3 | 1.204478476 |
| Interleukin-1 receptor-associated kinase 1 binding protein 1 | IRAK1BP1 | 1.19008679 |
| jumonji domain containing 1C | JMJD1C | 1.215369134 |
| jumonji domain containing 1C | JMJD1C | 1.43887561 |
| jumonji domain containing 1C | JMJD1C | 1.495380083 |
| jumonji domain containing 2B | JMJD2B | 1.536108405 |
| jumonji domain containing 3 | JMJD3 | 1.37485367 |
| jumonji domain containing 3 | JMJD3 | 1.100950746 |
| jumonji, AT rich interactive domain 1B | JARID1B | 1.47753956 |
| jumonji, AT rich interactive domain 1B | JARID1B | 1.090644346 |
| leukemia inhibitory factor receptor alpha | LIFR | 2.047338786 |
| leukocyte immunoglobulin-like receptor, subfamily A (without TM domain), | LILRA3 | 1.576089641 |
| member 3 | | |
| leukocyte receptor cluster (LRC) member 10 | LENG10 | 1.113443444 |
| Leukocyte-derived arginine aminopeptidase | LRAP | 1.00862931 |
| Lymphocyte antigen 86 | LY86 | 1.529616871 |
| lymphocyte antigen 9 | LY9 | 1.460881474 |
| macrophage scavenger receptor 1 | MSR1 | 1.291086986 |
| major histocompatibility complex, class I, A | HLA-A | 1.032578347 |
| Major histocompatibility complex, class I, A | HLA-A | 1.271717354 |
| major histocompatibility complex, class I, C | HLA-C | 1.450979765 |
| Major histocompatibility complex, class II, DR beta 1 | HLA-DRB1 | 1.232376825 |
| major histocompatibility complex, class I-related | MR1 | 2.006904213 |
| mesoderm induction early response 1, family member 3 | MIER3 | 1.869872636 |
| Mitogen activated protein kinase binding protein 1 | MAPKBP1 | 1.704950093 |
| mitogen-activated protein kinase 6 | MAPK6 | 1.22894334 |
| mitogen-activated protein kinase kinase kinase 12 | MAP3K12 | 1.109430463 |
| mitogen-activated protein kinase kinase kinase 13 | MAP3K13 | 1.060908844 |
| mitogen-activated protein kinase kinase kinase 7 interacting protein 3 | MAP3K7IP3 | 1.927650493 |
| Mitogen-activated protein kinase kinase kinase 8 | MAP3K8 | 1.352107524 |
| mitogen-activated protein kinase kinase kinase kinase 1 | MAP4K1 | 1.011528282 |
| mitogen-activated protein kinase kinase kinase kinase 1 | MAP4K1 | 1.050878193 |
| mitogen-activated protein kinase kinase kinase kinase 4 | MAP4K4 | 3.185779778 |
| mitogen-activated protein kinase-activated protein kinase 2 | MAPKAPK2 | 1.133827301 |
| Natural killer-tumor recognition sequence | NKTR | 1.891894783 |
| Nuclear factor I/A | NFIA | 2.086880166 |
| Nuclear factor I/A | NFIA | 1.331265556 |
| nuclear factor I/B | NFIB | 2.855325857 |
| nuclear factor I/C (CCAAT-binding transcription factor) | NFIC | 1.040283227 |
| nuclear factor I/C (CCAAT-binding transcription factor) | NFIC | 1.075135812 |
| Nuclear factor of activated T-cells, cytoplasmic, calcineurin-dependent 2 | NFATC2 | 1.075703654 |
| nuclear factor of kappa light polypeptide gene enhancer in B-cells inhibitor, | NFKBIA | 1.445207147 |
| alpha | | |
| nuclear factor of kappa light polypeptide gene enhancer in B-cells inhibitor- | NFKBIL1 | 1.464679525 |
| like 1 | | |
| platelet factor 4 variant 1 | PF4V1 | 1.499042181 |
| Platelet-activating factor acetylhydrolase, isoform Ib, alpha subunit 45 kDa | PAFAH1B1 | 1.134472215 |
| pre-B-cell colony enhancing factor 1 | PBEF1 | 1.085724086 |
| pre-B-cell colony enhancing factor 1 | PBEF1 | 1.520748539 |
| pre-B-cell colony enhancing factor 1 | PBEF1 | 1.55564941 |
| Pre-B-cell leukemia transcription factor 1 | PBX1 | 2.308702468 |
| pregnancy specific beta-1-glycoprotein 9 | PSG9 | 1.170493042 |
| Pregnancy-associated plasma protein A, pappalysin 1 | PAPPA | 1.521479019 |
| Prematurely terminated mRNA decay factor-like | LOC91431 | 2.107960171 |
| RAB GTPase activating protein 1-like | RABGAP1L | 1.653604709 |
| RAB18, member RAS oncogene family | RAB18 | 1.089188925 |
| RAB22A, member RAS oncogene family | RAB22A | 2.683053813 |
| RAB3 GTPase activating protein subunit 1 (catalytic) | RAB3GAP1 | 2.001733978 |
| RAB3 GTPase activating protein subunit 1 (catalytic) | RAB3GAP1 | 1.156175558 |
| RAB3 GTPase activating protein subunit 2 (non-catalytic) | RAB3GAP2 | 2.85334457 |
| RAB39B, member RAS oncogene family | RAB39B | 2.331854523 |
| RAB6A, member RAS oncogene family | RAB6A | 1.187513568 |
| RAB7B, member RAS oncogene family | RAB7B | 1.879994311 |
| RAB7B, member RAS oncogene family | RAB7B | 2.288912264 |
| Ras homolog gene family, member F (in filopodia) | RHOF | 2.138919058 |
| ras homolog gene family, member F (in filopodia) | RHOF | 1.071453498 |
| ras homolog gene family, member F (in filopodia) | RHOF | 1.142844537 |
| Ras homolog gene family, member H | RHOH | 2.285979964 |
| Ras homolog gene family, member H | RHOH | 1.231611795 |
| ras homolog gene family, member J | RHOJ | 2.964182828 |
| Ras homolog gene family, member T1 | RHOT1 | 1.37769877 |
| ras homolog gene family, member T1 | RHOT1 | 3.864365453 |
| ras responsive element binding protein 1 | RREB1 | 1.904794922 |
| Ras-associated protein Rap1 | RBJ | 2.652955528 |
| RasGEF domain family, member 1B | RASGEF1B | 3.324205937 |
| Ras-related GTP binding D | RRAGD | 1.371168629 |
| Response gene to complement 32 | RGC32 | 3.219990187 |
| response gene to complement 32 | RGC32 | 1.122371034 |
| signal transducer and activator of transcription 6, interleukin-4 induced | STAT6 | 1.148289999 |
| similar to bovine IgA regulatory protein | LOC492311 | 1.941490726 |
| similar to melanoma antigen family B, 18 /// | LOC653687 /// | 1.546018106 |
| opensimilar to chromosome X reading frame 50 | LOC729488 | |
| SUMO1/sentrin specific peptidase 6 | SENP6 | 1.749225224 |
| suppressor of cytokine signaling 1 | SOCS1 | 1.50931516 |
| Suppressor of cytokine signaling 7 | SOCS7 | 1.740525433 |
| T cell receptor alpha locus | TRA@ | 2.586569015 |
| T cell receptor alpha locus /// T-cell receptor active alpha-chain V-region (V- | TRA@ | 2.144132659 |
| J-C) mRNA, partial cds, clone AE212 | | |
| T-cell activation GTPase activating protein | TAGAP | 1.028883853 |
| T-cell activation NFKB-like protein | TA-NFKBH | 1.169292627 |
| T-cell lymphoma breakpoint associated target 1 | TCBA1 | 1.36158604 |
| T-cell receptor alpha, clone PPN82 | — | 1.334436033 |
| t-complex-associated-testis-expressed 3 | TCTE3 | 1.901540545 |
| TCR V-alpha w31 | — | 1.331818746 |
| TGFB-induced factor (TALE family homeobox) | TGIF | 1.018864234 |
| TNF receptor-associated factor 1 | TRAF1 | 1.896467635 |
| TNF receptor-associated protein 1 | TRAP1 | 2.320740104 |
| tolloid-like 1 | TLL1 | 1.734254297 |
| TP53 activated protein 1 | TP53AP1 | 1.674672894 |
| TPTE and PTEN homologous inositol lipid phosphatase pseudogene | LOC374491 | 1.355841886 |
| TRAF family member-associated NFKB activator | TANK | 1.233971012 |
| tumor necrosis factor receptor superfamily, member 10d, decoy with | TNFRSF10D | 1.055008839 |
| truncated death domain | | |
| tumor necrosis factor receptor superfamily, member 12A | TNFRSF12A | 4.407920874 |
| tumor necrosis factor receptor superfamily, member 18 | TNFRSF18 | 1.932110795 |
| tumor necrosis factor receptor superfamily, member 25 | TNFRSF25 | 2.333313996 |
| tumor necrosis factor, alpha-induced protein 3 | TNFAIP3 | 2.349930224 |
| Tumor protein D52 | TPD52 | 1.723222742 |
| tumor protein p53 inducible nuclear protein 2 | TP53INP2 | 1.765688824 |
| tumor suppressor candidate 3 | TUSC3 | 4.113431196 |
| tumor suppressor candidate 3 | TUSC3 | 1.2331631 |
| v-kit Hardy-Zuckerman 4 feline sarcoma viral oncogene homolog | KIT | 1.200046052 |
| v-maf musculoaponeurotic fibrosarcoma oncogene homolog (avian) | MAF | 1.099277019 |
| v-ral simian leukemia viral oncogene homolog A (ras related) | RALA | 1.003156349 |
| v-rel reticuloendotheliosis viral oncogene homolog (avian) | REL | 1.209415189 |

The present results are unprecedented for AIDS immunotherapy. Based on these results, the H-M is well positioned to make significant and novel contributions to AIDS immunotherapy.

Example embodiments of the methods and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

What is claimed:

1. A method of treating a viral infection comprising:
applying ultraviolet light to a blood sample containing a viral particle
stimulating the subject immune system to activate potent CMI and other immune responses against the virus.

2. The method according to claim 1, wherein the CMI activation further decreases viral load.

3. The method according to claim 1, wherein said CMI activation further reduces cellular inflammation associated with active viral infection.

4. The method according to claim 1, wherein said CMI activation further inhibits virus-induced inflammation.

5. The method according to claim 1, wherein said CMI activation is represented by cytokine activation

6. The method according to claim 1, wherein the said virus is an RNA virus.

7. The method according to claim 6, were in the said RNA virus is virus selected from a group consisting of Influenza and HIV

8. The method according to claim 1, wherein a wavelength of the ultraviolet light is in the range of 200 nm to 400 nm.

9. A method of treating HIV infection, comprising:
applying ultraviolet light to a blood sample containing an HIV particle
stimulating the subject immune system to activate potent CMI and other immune responses against the HIV virus.

10. The method according to claim 9, wherein HIV is HIV-1.

11. The method according to claim 9, wherein HIV is a HIV-2.

12. The method according to claim 9, wherein said CMI activation further decreases HIV viral load.

13. The method according to claim 9, wherein said CMI activation further reduces cellular inflammation associated with active HIV infection.

14. The method according to claim 9, wherein said CMI activation further results in inhibiting HIV virus-induced inflammation.

15. The method according to claim 9, wherein said CMI activation further results in cytokine activation.

16. The method according to claim 9, wherein the said cytokine is selected from a group consisting of IL1, IL2, IL3, IL4, IL5, IL6, IL7, IL8, IL9, IL10, IL11 and IL12.

17. The method according to claim 9 wherein the activation of said CMI further limits the scope and severity of clinical disease associated with a response to the HIV virus by a host regardless of antigenic sub-type of the virus or susceptibility to anti-viral medication.

18. The method according to claim 9, wherein a wavelength of the ultraviolet light is in the range of 200 nm to 400 nm.

19. A method of treating influenza infection, comprising:
applying ultraviolet light to a blood sample containing an influenza viral particle
stimulating the subject immune system to activate potent cell mediated immunity (CMI) and other immune responses against the influenza virus.

20. The method according to claim 19, wherein said CMI activation further decreases influenza viral load.

21. The method according to claim 19, wherein said CMI activation further reduces respiratory distress associated with active influenza infection.

22. The method according to claim 19, wherein said CMI activation further inhibits influenza-induced inflammation.

23. The method according to claim 19 wherein the activation of said CMI further limits scope and severity of clinical disease associated with a response to the influenza virus by a host regardless of antigenic sub-type of the influenza virus or susceptibility to anti-viral medication.

24. The method according to claim 19, wherein a wavelength of the ultraviolet light is in the range of 200 nm to 400 nm.
